(19) United States
(12) Patent Application Publication
(10) Pub. No.: US 2023/0329591 A1
(43) Pub. Date: Oct. 19, 2023

(54) SYSTEMS AND METHODS FOR AUTOMATED BIOLOGICAL FLUID COLLECTION

(71) Applicant: JABER MEDICAL, PLLC, West University Place, TX (US)

(72) Inventors: Noor Mahmoud Jaber, West University Place, TX (US); David Wong, West University Place, TX (US)

(21) Appl. No.: 18/300,346

(22) Filed: Apr. 13, 2023

Related U.S. Application Data
(60) Provisional application No. 63/362,923, filed on Apr. 13, 2022.

(51) Int. Cl. A61B 5/155; A61B 5/145; A61B 5/15

(52) U.S. Cl. CPC A61B 5/1427; A61B 5/14507; A61B 5/14503; A61B 5/150221; A61B 5/150343; A61B 2560/0266

(57) ABSTRACT

Provided here are systems and methods for the collection of a biological fluid sample from a subject. These automated systems can be programmed to extract a particular amount of biological fluid samples on a periodic basis from a patient.

### TECHNICAL FIELD

The present disclosure relates to systems and methods for the collection of a biological fluid from a subject. Provided herein are automated systems that facilitate biological fluid extraction from a patient under minimal supervision.

### BACKGROUND

In view of the increasing worldwide shortage of and ever-burgeoning demand on health care workers, there is a corresponding requirement for temporally-effective health care solutions for patients and other care recipients receiving on-site care (including hospitals, nursing homes, and convalescent centers), or in-home care. For many individuals, an invariable aspect of their requisite caregiving is based on physicochemical assays and specimen collection, such as routine blood collection, and/or the delivery of medicaments, nutrients, and other fluids via infusion processes.

However, most commercially-available specimen collection and infusion apparatuses require monitoring and vigilance, thereby enhancing the demands on both on-site and remote caregivers. In addition, such monitoring necessarily increases both the cost and associated liability for health care facilities and caregivers.

### SUMMARY

Accordingly, there exists a need for addressing the foregoing inequities and related issues. In addressing aspects of the aforementioned and other challenges in the art, the present disclosure is directed to methods and systems for biological fluid collection and for the infusion of medicaments and other fluids to a patient's body. The disclosed systems and methods, in certain embodiments, can reduce time and/or staffing requirements for specimen collection or medicament infusion, and can ensure proper operations of the systems, updates to the patient's electronic medical record (EMR) and/or electronic health record (EHR) as necessary, and notification to the caregivers of potential issues associated with specimen collection(s) and/or infusion(s).

In certain aspects, the present disclosure is directed to a system for collecting a biological fluid sample. One such system includes a sampling assembly in fluid connection with an intravenous (IV) catheter and configured to collect a biological fluid sample, a valve assembly having one or more actuators and a corresponding number of channels configured to transport the biological fluid sample to a sample collection device, and a controller in signal communication with the sampling assembly and the valve assembly. In certain embodiments, the valve assembly can have two or more actuators and a corresponding number of channels configured to transport the biological fluid sample to the sample collection device. This controller has one or more processors and a memory having computer-readable instructions that, when executed by the processor, cause the processor to (a) transmit a signal to initiate collection of a predetermined volume of the biological fluid sample by the sampling assembly, (b) extract the biological fluid sample using the sampling assembly, and (c) transport the biological fluid sample to the sample collection device using one of the number of channels of the valve assembly. In embodiments, the sampling assembly includes a lancing apparatus configured to collect one or more biological fluid samples. In certain embodiments, the system includes a user interface to receive one or more inputs regarding collection and transport of the biological fluid sample, such as time, amount, or frequency of collection. The user interface can be a touch screen display.

In further aspects, the actuator may include one or more servomotors or servo motors. In certain aspects, the computer readable instructions further cause the processor to transmit a signal to a user device in signal communication with the controller to indicate completion of collection of the biological fluid sample. In other embodiments, the computer readable instructions further cause the processor to send signals to flush and reset the sampling assembly and the valve assembly for initiation of a collection of a second biological fluid sample.

For instance, the disclosed system may readily accommodate a patient requiring a single biological fluid sample collection or multiple biological fluid sample collections for assaying or evaluating any number of biomedically-relevant compounds, which in non-limiting aspects may be blood, cerebrospinal fluid, blood solutes, interstitial fluid, or a fraction of a biological fluid. Moreover, the system may readily accommodate multiple patients for whom one or more biological fluid sample collections may be necessary. In related aspects, the controller (as well as components of the controller, and/or devices in signal communication with the controller) may be controlled by a caregiver or a system administrator in a different location with respect to the system. For instance, the controller is capable of beneficially facilitating biological fluid sample collection remotely by authorized individuals such as a caregiver or system administrator. In certain embodiments, other fluids may be infused to the patient through the system, such as lipids, electrolytes, glucose, pharmaceutical compounds, supplements, and metabolites.

In some embodiments, the controller includes a microcontroller in signal communication with one or more electronic devices that can be a portable electronic device, a portable computer, a desktop computer, a cloud based server, or a database server. For example, a patient may be subjected to one or more biological fluid sample collections, for instance, one or more blood draws, after which the system may perform one or more of (1) alerting a caregiver and/or a system administrator that the one or more biological fluid sample(s) have been collected; and (2) updating the patient's EMR and/or EHR. The caregiver or system administrator may be alerted using one or more of a portable electronic device, such as a smartphone or a laptop, a desktop computer, a server in signal communication with the system. The system can be one or more of a cloud based server, a database server, any dedicated server capable of data file storage, including any non-transitory machine-readable storage medium.

Conversely, in the event that the one or more biological fluid samples are not properly or timely collected, for instance, if a patient is not in operable connection with the system, if collection of the sample is not complete due to resistance, occlusions, pressure dissipation, and related issues, within one or more components of the system, of air, moisture, and related complications, are present in the line, and/or if the system is not otherwise operable, a caregiver, system administrator or related personnel may be alerted through one or more of a portable electronic device, a portable computer, a desktop computer, or similar equipment, and the patient's EMR and/or EHR may be updated using a cloud based server, a database server, or related data storage medium.

In further aspects, the controller is in signal communication with a displayable graphical user interface (GUI). For instance, the disclosed system can include a displayable monitor, such as a monitor capable of being connected to and operable to display an interface, for instance, a GUI, with its file array and requirements housed on a computer, laptop, tablet, smartphone and related devices. In additional embodiments, the GUI may be touchably responsive to a user, such as a caregiver or a system administrator, for displaying information of interest to the user and/or for controlling one or more aspects of the system. In related aspects, the GUI is associable with the disclosed system and may, in aspects, confer a caregiver or a system administrator, with the ability to executably control aspects of the system, including but not limited, to biological fluid sample collection volume(s), biological fluid sample collection time(s), the rate at which one or more biological fluid samples are introduced into a sample collection device. The GUI may be further enabled, in certain embodiments, to facilitate the display of at least a portion of one or more of a patient's information, prescription records, EMR, or EHR.

In certain embodiments, the intravenous (IV) catheter is optionally attached to a subject. In additional aspects, a central venous catheter (CVC), such as a peripherally-inserted central catheter, a gastric tube, a nasogastric tube, a rectal tube, or additional tube, a port, or a line, capable of being in fluid communication with a patient may be utilized in accordance with the system and methods of the present disclosure. For instance, the intravenous (IV) catheter may be attached to a medical tube or line in fluid contact with the subject which may include, in embodiments, a medical line or tube that has previously been inserted into a patient, or a medical tube or line in direct or indirect fluid contact with the inserted medical line or tube. During the infusion of an IV medication, the device can also clamp off and restart that line as needed. For example, if a particular medication, such as a chemotherapeutic agent or an immunotherapeutic agent, is not able to run through the specimen collection circuit, this alternate configuration would allow it to run in a multi-lumen IV catheter, such as a peripheral or a central line.

In certain embodiments, the valve assembly can include one or more valves, and in certain aspects may optionally include a rotatable multichannel valve. In still further aspects, the one or more actuators of the valve assembly are individually selected from a rotary actuator, a linear actuator or a combination thereof, each of which may be individually in signal communication with the controller. Alternatively, the one or more actuators of the valve assembly may be collectively controlled by the controller. In embodiments, the valve assembly can have a plurality of channels. For example, the valve assembly may contain two channels capable of biological fluid sample collection and one channel inoperable for biological fluid sample collection where, in certain aspects, the third channel may comprise a syringe capable of injecting or flowing a fluid such as air, a gas, a buffer, a saline solution or a cleaning fluid for sufficiently flushing the system following the completion of one or more biological fluid sample collections, including the completion of a biological fluid collection cycle as further described herein.

The biological fluid sample(s) that may be collected in accordance with the disclosed system and methods are variable and can depend on the patient, disease, or treatment regimen. For instance, in some aspects the biological fluid sample is one or more of blood, cerebrospinal spinal, blood solutes, interstitial fluid, or a fraction of a biological fluid. Additionally, the biological fluid sample may, in particular embodiments, comprise or consist essentially of blood. However, the system and methods of the present disclosure can collect any biological fluid capable of being fluidly deposited into a sample collection device as described herein, including samples capable of being collected through a catheter, line, tube, and/or port, and deposited into a sample collection device accordingly. In particular embodiments, a biological fluid sample collection is performed under sterile conditions. In certain embodiments, other fluids may be infused to the patient through the system, such as lipids, electrolytes, glucose, pharmaceutical compounds, supplements, and metabolites.

While the volume of a biological fluid sample to be collected may be adjusted based on myriad factors such as the type of tests or assays to which the sample will be subjected, including the present health of the patient, in certain aspects the collected biological fluid sample volume is generally in a range of about 1 microliter (μl) to about 20 milliters (ml), including about 1 (ml) to about 20 ml, including a range of about 5 ml to about 15 ml, including about 10 ml. In related embodiments, biological fluid sample collection occurs over a predetermined time period in a range of about 30 seconds to about 12 hours (about 720 minutes), such as in a range of about 1 minute to about 6 hours (about 360 minutes), about 2 minutes to about 4 hours (about 240 minutes), about 3 minutes to about 2 hours (about 120 minutes), and about 4 minutes to about 1 hour (about 60 minutes), including about 5 minutes. For example, a single biological fluid sample may be collected via a relatively short collection cycle, for instance, about 5-10 minutes, for performing a routine assay or an expedited assay on a fluid sample such as a blood sample. In further aspects, two (or more) biological fluid samples may be collected over a more temporally extensive period, for instance, a time period of about 4 hours (about 240 minutes), where an initial biological fluid sample reading is desired at a first time point of about 0 minutes and a subsequent biological fluid sample reading at a second time point of about 240 minutes.

In some aspects, the present disclosure provides for methods for collecting a biological fluid sample from a subject. The method includes the following steps: (1) propagating a signal from a controller to a sampling assembly and a valve assembly for initiating collection of a biological fluid sample; (2) extracting a biological fluid sample from an intravenous (IV) catheter attached to the subject using the sampling device; (3) transporting the biological fluid sample using the valve assembly having one or more actuators in signal communication with a controller; and (4) depositing the collected biological fluid sample in a sample collection device.

In additional embodiments, the method may further include transmitting a signal from the controller to a user device in signal communication with the controller to indicate completion of collection of the biological fluid sample. In further aspects, the method may include flushing and resetting the sampling assembly and the valve assembly for initiating a subsequent biological fluid sample collection cycle. In certain embodiments, the propagation of the signal from a controller for commencing the methodology is performed by an electronic device in signal communication with the controller selected from a portable electronic device, a portable computer, a desktop computer, a cloud based server, and a database server.

### BRIEF DESCRIPTION OF DRAWINGS

The foregoing embodiments and other features, aspects, and advantages of the disclosure will become better understood with regard to the following descriptions, claims, and accompanying drawings. It is to be noted, however, that the drawings illustrate only several embodiments of the disclosure and, therefore, are not to be considered limiting of the breadth and scope of the disclosure.

FIGS. 1A and 1B are illustrations of various aspects of a biological fluid sample collection system, according to certain embodiments of the disclosure.

FIG. 2 is an illustration of various aspects of a method for biological fluid sample collection, in accordance with various aspects of the present disclosure.

FIGS. 3A and 3B are illustrations of various aspects of a rotatable multichannel valve for use in accordance with certain embodiments of the disclosure.

FIG. 4 is an illustration of the external housing of the biological fluid sample collection system with a graphic user interface, according to certain embodiments of the disclosure.

FIG. 5 is illustration of a biological fluid sample collection system, according to certain embodiments of the disclosure.

While particular or specific features and embodiments may be depicted in certain individual drawings and absent in other drawings, the skilled artisan will appreciate that the features and embodiments of the drawings may be partially or completely combined in accordance with various teachings of the instant disclosure.

### DETAILED DESCRIPTION

So that some of the various features and advantages of the embodiments and aspects of the biomedical specimen collection and infusion systems disclosed herein, as well as methods related thereto, which will become apparent, may be understood in greater detail, a more particular description of embodiments of systems and methods briefly summarized above may be had by reference to the following detailed description of embodiments thereof, in which one or more are further illustrated in the appended drawings, which form a part of this specification. It is to be noted, however, that the drawings illustrate only various embodiments of the systems and methods disclosed herein and are therefore not to be considered limiting of the breadth and scope of the disclosed systems and methods.

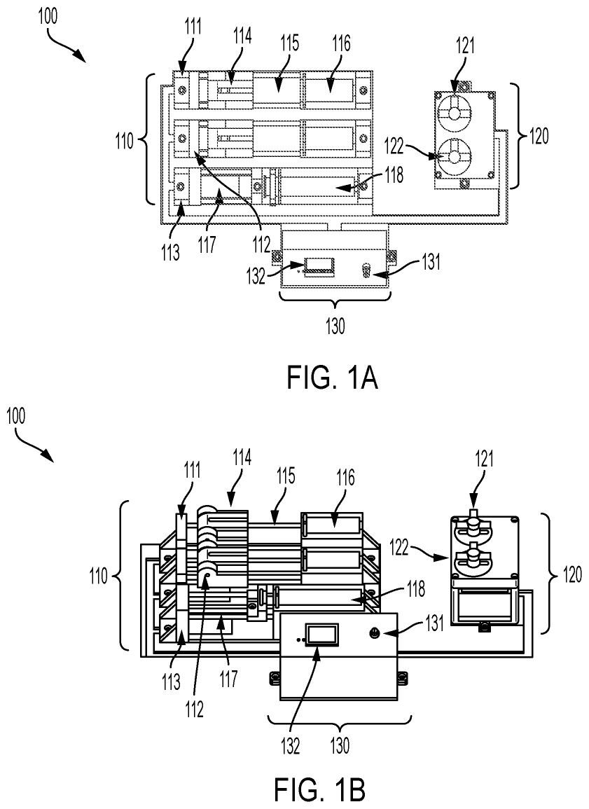

In one or more embodiments, as illustrated in FIGS. 1-3, the present disclosure is directed to systems for collecting biological fluids, systems for performing infusions of medicaments or nutrients, as well as methods related thereto, including methods for collecting and administering specimens, fluids, and samples, as appropriate, as described herein.

In certain aspects, the present disclosure is directed to a system comprising, inter alia, components including a sampling assembly, a valve assembly, and a controller in signal communication with the sampling assembly and the valve assembly. As used herein, “signal communication” refers to electric communication, such as hard wiring two components together or wireless communication, as understood by those skilled in the art. In addition, wireless communication through one or more methods of communication such as WiFi®, Bluetooth®, Zigbee, or forms of communications, including near-field communications, may be employed in accordance with the present disclosure. In addition, signal communication may include one or more intermediate controllers or relays disposed between elements that are in signal communication with one another.

As used herein, a “processor” may include, for example one processor or multiple processors included in a single device or distributed across multiple computing devices. The processor may be at least one of a central processing unit (CPU), a semiconductor-based microprocessor, a graphics processing unit (GPU), a field-programmable gate array (FPGA) to retrieve and execute instructions, a real time processor (RTP), other electronic circuitry suitable for the retrieval and execution instructions stored on a machine-readable storage medium, or a combination thereof.

The term “non-transitory machine-readable storage medium,” as used herein, may be any electronic, magnetic, optical, or other physical storage apparatus to contain or store information such as executable instructions, data, and the like. For example, any machine-readable storage medium described herein may be any of random access memory (RAM), volatile memory, non-volatile memory, flash memory, a storage drive (such as a hard drive), a solid state drive, any type of storage disc, and the like, or a combination thereof.

As shown in FIG. 1, which provides for certain aspects of a biological fluid sample collection system 100 in accordance with the present disclosure, one or more sample collection devices (not shown) may initially be loaded into the channels of the sampling assembly 110. This sampling assembly can include individual sample actuators 111, 112, 113 and, in embodiments, is in fluid connection with an intravenous (IV) catheter (not shown). In embodiments, sample actuators 112 and 113 are operably arranged and available for biological fluid sample collection, while actuator 114 is operably arranged and available to flush the system of residual fluids, materials, and the like, following a biological fluid sample collection cycle as described herein. One or more sample collection devices may be secured into sample actuators 111, 112 via, in embodiments, sample collection device holder 114 of sample actuator 111 following loading of the same within the sample actuator by introduction of the sample collection device into sample collection device channel 115. This biological fluid sample collection system 100 can include a single actuator to coordinate biological fluid sample collection, flushing of the system, and infusion of a fluid, such as electrolytes, glucose, pharmaceutical compounds, supplements, and metabolites.

In aspects, sample collection devices may be loaded into a sample collection device holder, such as sample collection device holder 114, by the initial, reversible pivoting of sample collection device holder to a position of, for example, about 90° with respect to the plane of sample collection device channel 115, followed by pivoting of sample collection device holder 114 to a co-planar position with respect to sample collection device channel 115. In additional embodiments, sample actuator 113 is loading with a syringe of suitable volume, for example, a 10 ml syringe (not shown), that is loaded into syringe holder 118 via introduction into syringe channel 117.

In embodiments, the syringe is suitably and stably supported by syringe holder 118 such that brackets, braces, and related equipment for further securing the syringe are beneficially not required. In related aspects, only the syringe plunger (not shown) of the syringe is movable or repositionable, for example, the syringe barrel or body (not shown) is immovable upon its introduction into syringe holder 118 and the syringe plunger is reversibly withdrawn and re-inserted into the syringe barrel or body as necessary and in accordance with, flushing the system prior to and/or following biological fluid sample collection. In embodiments, the syringe plunger is withdrawn from the syringe barrel or body prior to the initiation of biological fluid sample collection.

Following the loading and positioning of the sample collection devices and the syringe, controller 130 transmits a signal to sampling assembly 110 to initiate biological fluid sample collection and provide a predetermined collection volume for the biological fluid. Initiation of the biological fluid sample collection, in further aspects, may further initiation of the operability of the graphical user interface (GUI), such that the user may actively control or specify biological fluid sample collection conditions, including but not limited to collection times, sample collection velocity or speed, pressure conditions, sample collection device size (including volume and/or device length), frequency of sample collection, and further conditions as described herein. The GUI may, in embodiments, be displayed on a standalone monitor or display such as interface 402, and/or it may be displayable or housed on a computer, laptop, tablet, smartphone and related devices as further disclosed herein.

Following the initiation of biological fluid sample collection, one or more sample collection devices are loaded into a sample collection device holder, such as sample collection device holder 114 of sample actuator 111, with sample actuator 112 similarly capable of accommodating the loading of a sample collection device via an associated sample collection device holder. In related aspects, two sample collection devices loaded into sample actuators 111, 112 are sequentially and respectively advanced towards a sample collection device receiver. For instance, sample actuator 111 initially advances its sample collection device holder towards its sample collection device receiver 116, followed by sample actuator 112 subsequently advancing its sample collection device holder towards an associated sample collection device receiver.

Upon advancement of a sample collection device by a sample actuator into its respective sample collection device holder, for instance, by sample actuator 111 advancing its sample collection device towards its respective sample collection device receiver 116, a sample is fluidly introduced into the sample collection device via active pumping of the sample, for instance, from a patient (or, in alternative embodiments, a biological fluid sample previously drawn from the patient) in fluid connection with an IV, a catheter, and/or a port, using valve assembly 120, which is in fluid connection with the sample collection device through one or more tubes and/or hoses, and one or more associated connectors including valves and stopcocks (not shown).

The completion of the collection of the first biological fluid sample initiates the subsequent introduction of sample collection device (not shown) residing in actuator 112 into its associated sample collection device receiver, wherein a biological fluid sample is introduced into the sample collection device of actuator 112 and its associated sample collection device receiver. Similar to the collection of the first biological fluid sample, the second biological fluid sample is fluidly introduced into the sample collection device via active pumping of the sample, for instance, from a patient or patient sample in fluid connection with an IV, a catheter, a port, or related components, using valve assembly 120, which is in fluid connection with sample assembly 110, including the relevant components of sample assembly 110 as described herein, through one or more tubes and/or hoses, and one or more associated connectors including valves, stopcocks, and/or similar equipment (not shown), as further described below.

The completion of the collection of the second biological fluid sample initiates the subsequent flushing of biological fluid sample collection system 100, including any residual fluids, plasmas and/or additional residual material capable of being flushed from components in fluid contact with a flushable stream that is generated by depression of the syringe plunger into the syringe barrel or body (not shown). In related aspects, the system flushing may be performed by employing one or more of air, one or more gases, a buffer, a saline solution or a flushing or cleaning fluid for sufficiently flushing the system following the completion of one or more biological fluid sample collections. In embodiments, the fluids, plasmas, and/or residues that are flushed from the system, as well as the fluid(s) utilized in flushing the system, may be deposited or collected in a waste bin, a drain, or any receptacle capable of receiving the foregoing effluents. In further aspects, the syringe plunger may be reversibly returned to its initial position, including aspects where the syringe plunger is partially or fully withdrawn from the syringe barrel or body, and may optionally draw fluid(s) into the syringe barrel or body for additional system flushing, for beneficially performing one or more subsequent biological fluid sample collections.

As further shown in FIG. 1, valve system 120 may include multiple components capable of beneficially adjusting the pressure of biological fluid sample collection system 100 such that one or more biological fluid sample collection cycles may be performed. In some aspects, and as shown in FIG. 1, valve system 120 can include two or more valve actuators, such as valve actuators 121, 122. In embodiments, two or more valve actuators may be operably arranged and connected to two or more corresponding tubes, hoses, and/or related equipment, and two or more associated connectors including valves, stopcocks, and/or related equipment (not shown), such that valve system 120 is in operable and fluid connection with sample assembly 110.

For instance, in non-limiting aspects valve assembly 120 is in fluid connection with sample assembly 110 using a combination of standard 3-way stopcocks, each of which may be operably arranged in connection with valve actuators 121, 122 arranged such that for each standard 3-way stopcock (each of which includes 3 channels or ports) two channels or ports may be capable of fluidly connecting to or facilitating fluid connection between an IV, a catheter, a port, and/or similar components, and sampling assembly 110, including sample collection devices and/or a syringe (not shown) mounted therein for collecting a biological fluid sample. In this regard, the use of a 3-way stopcock may advantageously facilitate, in aspects, one or more of the rapid, controllable and/or sterile fluid connection between the biological fluid sample source, including an IV, a catheter, and a port, and the one or more sample collection devices of sampling assembly 110.

In some aspects, biological fluid collection system 100 further includes a controller, such as controller 130, which comprises and/or is in signal communication with a displayable graphical user interface (GUI), sampling assembly 110 and/or valve assembly 120. For instance, controller 130 may comprise or be in signal communication with a displayable monitor associated with, in non-limiting aspects, a computer, a laptop, a tablet, a smartphone, or other devices capable of receiving and rendering information from controller 130 for beneficially providing a caregiver and/or a system administrator the capacity to review and/or executably control aspects of the system including but not limiting to controlling the biological fluid sample collection volume(s), the biological fluid sample collection time(s), the biological fluid sample collection rate(s), and additional parameters.

In aspects, a GUI associated with controller 130 may be further enabled to display a patient's information following retrieval of the same, including but not limited to prescription records, EMR(s), and/or EHR(s), for optimizing patient care. In embodiments, controller 130 is equipped with one or more controllers, displays (which may be associated with a GUI or, alternatively, may present as a non-GUI display), such as controller dial 131 and controller display 132.

In embodiments, biological fluid sample collection system 100 may further be equipped with one or more sensors and/or cameras (not shown) for providing any necessary or desired surveillance of the system, for example, for maintaining patient safety, comfort and/or confidentiality. In related aspects, sensors and/or cameras for use in, in signal communication with, or proximal to biological fluid sample collection system 100 may communicate with one or more caregivers and/or system administrators capable of operating equipment including any one or more of monitors, CPUs, and other sensor controllers in signal communication with the sensors and/or cameras.

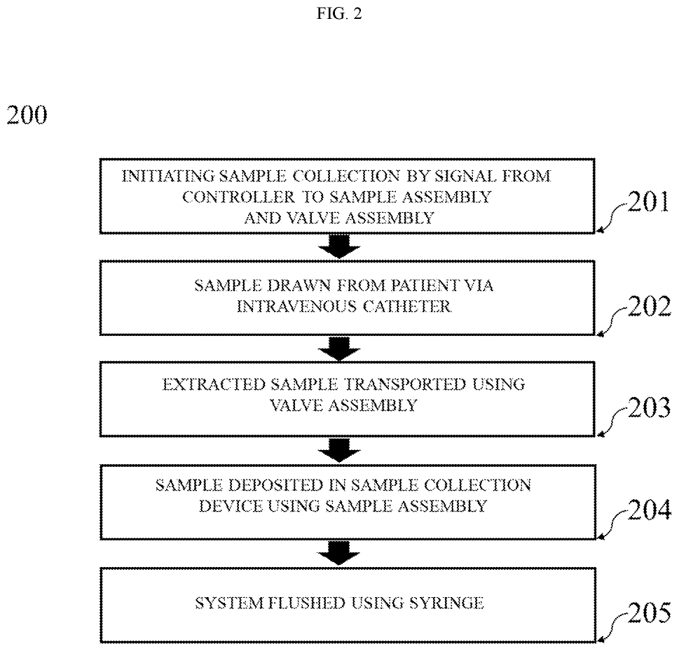

FIG. 2 illustrates a flow chart of certain aspects of a method 200 for the collection of a biological fluid sample, with reference to the relevant components exemplified in FIGS. 1A and 1B. In block 201, a method for biological fluid sample collection is initiated by propagation of a signal from controller 130 to sampling assembly 110 and valve assembly 120. The biological fluid sample is drawn or extracted from an intravenous (IV) catheter that is directly inserted into a patient or subject. In block 202, a tube or series of tubing in fluid connection with sampling assembly 110 and valve assembly 120 fluidly transfers the biological fluid sample from intravenous (IV) catheter to sampling assembly 110. As described above, the volume of and the extraction time for the biological fluid sample may be controlled, in aspects, by controller 130, for instance, using one or more dials in signal communication with controller 130, or using a graphical user interface (GUI) in signal communication with controller 130, and monitored via a display associated with controller 130 such as controller display 132 and/or a GUI.

At block 203, the extracted biological fluid sample is subsequently transported from sampling assembly 110 using valve assembly 120, where sampling assembly 110 comprises one or more individual sample actuators such as sample actuator 111. Sample actuator 111, in accordance with embodiments of the disclosed method, contains a sample collection device for deposition of an extracted biological fluid sample. At block 204, sampling assembly 110 transports the extracted biological fluid sample using valve assembly 120, which is equipped with one or more valve actuators such as valve actuators 121, 122 in fluid communication with sampling assembly 110 via one or more tubes or a series of interconnected tubing, and deposits the extracted biological fluid sample in a sample collection device. At block 205, one or more sample actuators such as sample actuator 113 may be equipped with a syringe for flushing one or more components, including every component of biological fluid sample collection system 100 as described in the foregoing, in accordance with the disclosed methodology.

In further aspects, a method for biological fluid sample collection system such as that depicted in 200 may optionally be performed using additional, optional equipment such as one or more sensors and/or cameras for best providing any necessary or desired surveillance of the system, including for best maintaining patient safety, comfort and/or confidentiality. The sensors and/or cameras may be directly incorporated into, may be in signal communication with, or may be proximal to the components associated with method for biological fluid sample collection 200, for instance, in aspects for advantageously communicating with one or more caregivers and/or system administrators, that may be operating any of the foregoing components for providing sufficient care and vigilance for a patient or subject.

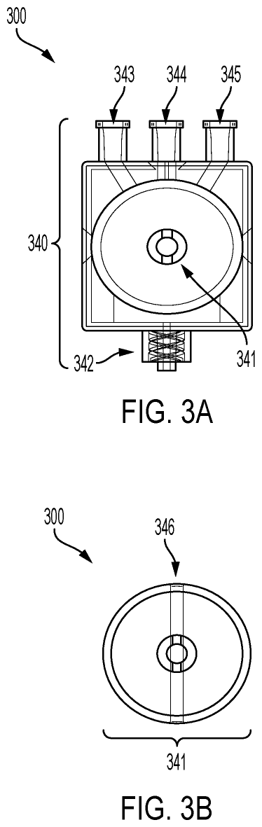

As will be appreciated by the skilled artisan, aspects of a biological fluid sample collection system such as biological fluid sample collection system 100 and biological fluid sample collection method 200 of FIGS. 1 and 2, respectively, may benefit from a single, multichannel valve, such as rotatable multichannel valve 300, in comparison with, for instance, a system equipped with multiple valve actuators and associated standard 3-way stopcocks, tubing, and/or hosing, as illustrated and/or described in the foregoing descriptions, including valve assemblies 120 and 220 of biological fluid sample collection systems 100 and 200 of FIGS. 1 and 2, respectively. For instance, as shown in FIG. 3A, a rotatable multichannel valve 300 may include a fluid valve port 342 for facilitating direct fluid contact with an IV, a catheter, and/or a port, through one or more adjoining tubes or hoses (not shown) for drawing a biological fluid sample from a patient or sample vessel containing the biological fluid sample.

The rotational capacity of rotatable multichannel valve 300, which is endowed by a rotary element, such as valve rotary element 341, beneficially allows for the selectable deposition of a collected biological fluid sample into one or more sample collection devices associated with a system for biological fluid sample collection (not shown) in fluid connection with rotatable multichannel valve 300 through one or more adjoining tubes or hoses (not shown), including such tubes and/or hoses operably connected to fluid valve channels 343, 344, 345, each of which may be fluidly connected to a system for biological fluid sample collection, for instance, through a sampling assembly (not shown) as described in the foregoing. In another embodiment as shown in FIG. 3B, a valve rotary element channel 346 of rotatable multichannel valve 300 may introduce a biological fluid sample into a fluidly-adjoining system for biological fluid sample collection (not shown) using any one of fluid valve channels 343, 344, 345 for the introduction of a biological fluid sample to a sample collection device (not shown), for advantageously reducing the risk of corrupting the biological fluid sample through, for example, exposure to non-sterile conditions (such as ambient atmosphere conditions).

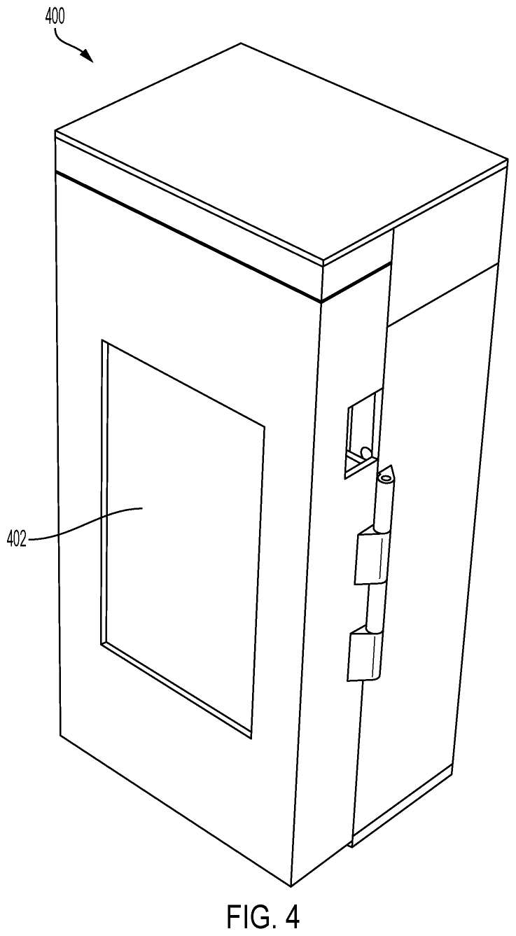

FIG. 4 is an illustration of the external housing of the biological fluid sample collection system 400 with a GUI 402. The GUI can be responsive to touch of a user, such as a caregiver or a system administrator, for displaying information of interest to the user and/or for controlling one or more aspects of the system. The GUI 402 is associable with the disclosed system and may, in aspects, confer a caregiver or a system administrator, with the ability to executably control aspects of the system, including but not limited, to biological fluid sample collection volume(s), biological fluid sample collection time(s), the rate at which one or more biological fluid samples are introduced into a sample collection device. Initiation of the biological fluid sample collection, in further aspects, may further initiation of the operability of the GUI 402, such that the user may actively control or specify biological fluid sample collection conditions, including but not limited to collection times, sample collection velocity or speed, pressure conditions, sample collection device size (including volume and/or device length), frequency of sample collection, and further conditions as described herein.

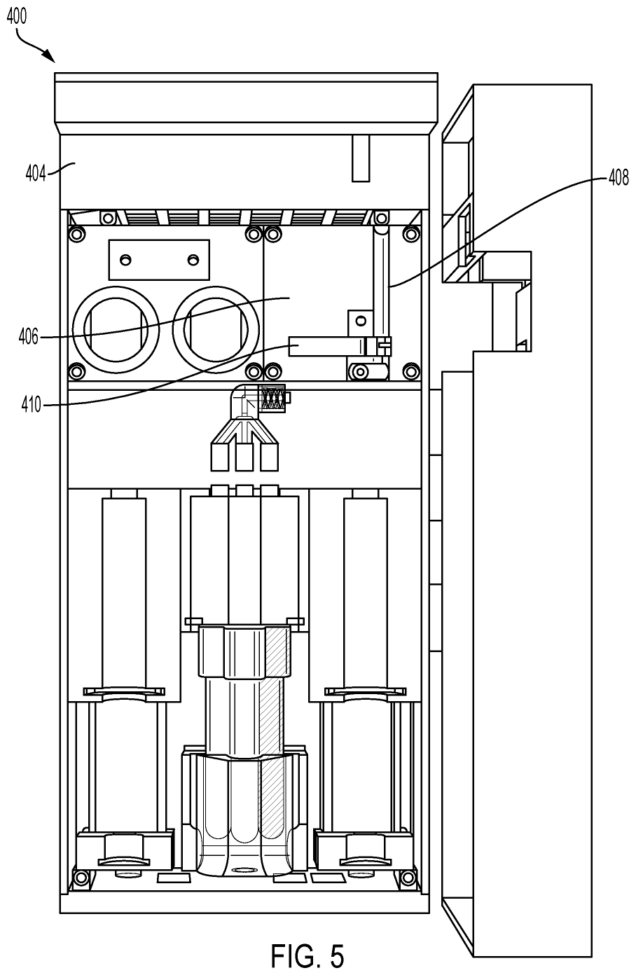

FIG. 5 is illustration of a biological fluid sample collection system 400, according to certain embodiments of the disclosure. The biological fluid sample collection system 400 includes an electronic housing 404. This system 400 includes an additional functional feature 406 housing a mechanism that allows for the clamping of an IV line, such as a servo motor. The system also includes an IV line 408 for infusion of a medication and a clamping mechanism 410. At any time during the infusion, the line can be clamped off and infusion can be delayed or stopped. The clamping mechanism can be a servo with horn to stop the infusion and restart that line as needed. For example, if a particular medication, such as a chemotherapeutic agent or an immunotherapeutic agent, is not able to run through the specimen collection circuit, this alternate configuration would allow it to run in a multi-lumen IV catheter, such as a peripheral or a central line.

Although specific terms and phrases are incorporated herein, any such terms and phrases are used in a descriptive sense only and not for the purposes of limitation. Embodiments of systems and methods for the collection of biological fluid sample have been described in considerable detail with specific reference to the illustrated embodiments and aspects of the present disclosure. However, it will be apparent to those of skill in the art that various modifications and changes may be employed within the spirit and scope of the embodiments of systems and methods as described in the foregoing specification, and such modifications and changes are to be considered equivalents and part of this disclosure.

What is claimed is:

1. A system for collecting a biological fluid sample, the system comprising:
a sampling assembly in fluid connection with an intravenous (IV) catheter and configured to collect a biological fluid sample;
a valve assembly having one or more actuators and a corresponding number of channels configured to transport the biological fluid sample to a sample collection device; and
a controller in signal communication with the sampling assembly and the valve assembly, the controller with one or more processors and a memory having computer-readable instructions that, when executed by the processor, cause the processor to transmit a plurality of signals to:
initiate collection of a predetermined volume of the biological fluid sample by the sampling assembly;
extract the biological fluid sample using the sampling assembly; and
transport the biological fluid sample to the sample collection device using the corresponding channel of the valve assembly.

2. The system of claim 1, wherein the computer readable instructions further cause the processor to transmit a signal to a user device in signal communication with the controller to indicate completion of collection of the biological fluid sample.

3. The system of claim 1, wherein the computer readable instructions further cause the processor to transmit a signal to flush and reset the sampling assembly and the valve assembly for initiation of a collection of a second biological fluid sample.

4. The system of claim 1, wherein the valve assembly comprises a rotatable multichannel valve.

5. The system of claim 1, wherein the one or more actuators of the valve assembly are individually selected from a rotary actuator, a linear actuator or a combination thereof.

6. The system of claim 1, wherein each of the actuators of the valve assembly are in individual signal communication with the controller.

7. The system of claim 1, wherein the valve assembly contains three actuators and three channels.

8. The system of claim 7, wherein the three channels of the valve assembly comprise two channels operable for biological fluid sample collection and one channel operable for system flushing.

9. The system of claim 1, wherein the biological fluid sample is blood or cerebrospinal fluid.

10. The system of claim 1, wherein the biological fluid sample consists essentially of blood.

11. The system of claim 1, wherein biological fluid sample collection is performed under sterile conditions.

12. The system of claim 1, wherein the predetermined volume is in a range of about 1 μl to about 20 ml.

13. The system of claim 1, wherein the biological fluid sample is collected over a predetermined time period ranging from about 30 seconds to about 12 hours.

14. A method for collecting a biological fluid sample from a subject, the method comprising:
propagating a signal from a controller to a sampling assembly and a valve assembly for initiating collection of a biological fluid sample;
extracting the biological fluid sample from an intravenous catheter attached to the subject using the sampling assembly;
transporting the biological fluid sample from the sampling assembly using the valve assembly having one or more actuators in individual signal communication with the controller; and
depositing the biological fluid sample in a sample collection device.

15. The method of claim 14, further comprising the step of transmitting a signal from the controller to a user device in signal communication with the controller to indicate completion of collection of the biological fluid sample.

16. The method of claim 14, further comprising the step of flushing and resetting the sampling assembly and the valve assembly.

17. The method of claim 14, wherein the biological fluid sample is blood or cerebrospinal fluid.

18. The method of claim 14, wherein the biological fluid sample consists essentially of blood.

19. The method of claim 14, wherein extracting and transporting of the biological fluid sample collection occurs under sterile conditions.

20. The method of claim 14, wherein the biological fluid sample is extracted at a predetermined volume ranging from about 1 μl to about 20 ml.
